(19) United States
(12) Patent Application Publication
(10) Pub. No.: US 2016/0317122 A1
(43) Pub. Date: Nov. 3, 2016

(54) IN-DEVICE FUSION OF OPTICAL AND INERTIAL POSITIONAL TRACKING OF ULTRASOUND PROBES

(71) Applicant: QUALCOMM Incorporated, San Diego, CA (US)

(72) Inventors: Ricardo Paulo dos Santos Mendonca, Seattle, WA (US); Patrik Nils Lundqvist, Encinitas, CA (US); Rashid Ahmed Akbar Attar, San Diego, CA (US); Rajeev Jain, Los Angeles, CA (US); Padmapriya Jagannathan, San Diego, CA (US)

(21) Appl. No.: 15/140,001

(22) Filed: Apr. 27, 2016

Related U.S. Application Data
(60) Provisional application No. 62/153,978, filed on Apr. 28, 2015.
(60) Provisional application No. 62/153,970, filed on Apr. 28, 2015.
(60) Provisional application No. 62/153,974, filed on Apr. 28, 2015.

(51) Int. Cl. A61B 8/00; A61B 8/08

(52) U.S. Cl. CPC A61B 8/4254; A61B 8/483; A61B 8/56; A61B 8/5223; A61B 8/5238; A61B 8/4483

(57) ABSTRACT

An apparatus for noninvasive medical ultrasonography includes one or more ultrasonic transducers, one or more inertial sensors, one or more optical sensors, and a processor communicatively coupled with the ultrasonic transducers, the inertial sensors and the optical sensors. The processor is configured to estimate a position of the apparatus based on a combination of signals received from the ultrasonic transducers, the inertial sensors and the optical sensors.

### CROSS-REFERENCE TO RELATED APPLICATIONS

This disclosure claims priority to U.S. Provisional Patent Application No. 62/153,978, filed on Apr. 28, 2015, entitled “AUTO-CONFIGURATION OF A DEVICE FOR ULTRASOUND IMAGING,” to Provisional Patent Application No. 62/153,970, filed on Apr. 28, 2015 and entitled “IN-DEVICE FUSION OF OPTICAL AND INERTIAL POSITIONAL TRACKING OF ULTRASOUND PROBES,” and to Provisional Patent Application No. 62/153,974, filed on Apr. 28, 2015 and entitled “OPTIMIZED ALLOCATION OF HETEROGENEOUS COMPUTATIONAL RESOURCES FOR ULTRASOUND IMAGING,” which are hereby incorporated by reference.

### TECHNICAL FIELD

This disclosure relates to an ultrasonography apparatus, and more particularly to techniques for improving the operability and functionality of the ultrasonography apparatus.

### DESCRIPTION OF THE RELATED TECHNOLOGY

High resolution ultrasonic imaging has been adapted for a large number of medical purposes. Traditionally, the ultrasonic imaging probe is a simple hand-held device that emits and receives acoustic signals. The device is connected by an electrical cable with a console or rack of equipment that provides control signals and power to the probe and that processes acoustic signal data received by the probe and forwarded to the console which processes the received data to produce viewable images of an anatomical feature of interest.

In the present disclosure, techniques are described for improving the operability and functionality of an ultrasonic imaging probe.

### SUMMARY

The systems, methods and devices of this disclosure each have several innovative aspects, no single one of which is solely responsible for the desirable attributes disclosed herein.

One innovative aspect of the subject matter described in this disclosure relates to an apparatus for ultrasonography that includes one or more ultrasonic transducers, one or more inertial sensors, one or more optical sensors, and a processor communicatively coupled with the one or more ultrasonic transducers, the one or more inertial sensors and the one or more optical sensors. The processor is capable of estimating a position of the apparatus based on a combination of signals received from the one or more ultrasonic transducers, the one or more inertial sensors and the one or more optical sensors.

In some examples, the estimating the position of the apparatus may include processing ultrasound image data from the one or more ultrasonic transducers and determining the position based on the processed ultrasound image data. In some examples, the ultrasound image data may include a series of 2-D image frames and the processed ultrasound image data may include a 3-D image. The processor may be configured to adjust at least one of the 2-D image frames in view of the determined position at a time of obtaining the at least one of the 2-D images.

In some examples, the ultrasound image data may include a series of 2-D image frames and the processed ultrasound image data may include a 3-D image of a first volume. The processor may be configured to determine, with regard to at least one of the 2-D image frames, whether the at least one of the 2-D image frames relates to the first volume or to a different volume.

In some examples, the optical sensor may be optically coupled with one or more optical wireless communication (OWC) emitters of an indoor positioning system. In some examples, the processor may be configured to correct drift error accumulation of the inertial sensors using the combination of signals.

In some examples, the processor may be configured to process image data acquired by one or both of the optical sensors and the ultrasonic transducers so as to select a plurality of landmarks. In some examples, the landmarks may include one or both of: (i) one or more points, edges or corners of ordinary surfaces, fixtures or objects of a room in which the apparatus is to be used to examine a subject; and (ii) one or more anatomical features of the subject, the anatomical features being selected from the group consisting of tissue surfaces, tissue boundaries and image texture of ordinary anatomical or pathological structures of the subject. In some examples, the processor may be configured to calculate the position of the apparatus with respect to the landmarks. In some examples, the processor may be configured to calculate a location of the subject or an anatomical feature of the subject.

In some examples, the processor may be configured to fuse the combination of signals using one or more of visual inertial odometry (VIO) techniques, simultaneous localization and mapping (SLAM) techniques, image registration techniques, or any combination thereof. In some examples, the processor may be configured to process ultrasound image data from the ultrasonic transducer and make a determination of the position of the apparatus from the processed ultrasound image data. In some examples, the processor may be configured to use the determination to provide, to an operator of the apparatus, one or more of: navigational guidance for movement of the imaging probe, notifications based on the determination, identification of anatomical features, identification of pathological structures or any combination thereof.

According to some implementations, a method for ultrasonography includes collecting image data of an environment in which an ultrasonography apparatus is to be operated. The ultrasonography apparatus includes one or more ultrasonic transducers, one or more inertial sensors, one or more optical sensors and a processor communicatively coupled with the one or more ultrasonic transducers, the one or more inertial sensors and the one or more optical sensors, the ultrasonography apparatus being configured to perform noninvasive medical ultrasonography. The method includes estimating, with the processor, a position of the apparatus using a combination of signals received from the one or more ultrasonic transducers, the one or more inertial sensors and the one or more optical sensors.

In some examples, the method includes fusing, with the processor, the combination of signals using one or more of visual inertial odometry (VIO) techniques, simultaneous localization and mapping (SLAM) techniques, image registration techniques, or any combination thereof.

In some examples, the image data may include outputs from one or both of the optical sensors and the ultrasonic transducers, the processor may be configured to process the image data so as to select a plurality of landmarks. The landmarks may include one or both of: (i) one or more points, edges or corners of ordinary surfaces, fixtures or objects of a room in which the apparatus is to be used to examine a subject; and (ii) one or more anatomical features of the subject, the anatomical features being selected from the group consisting of tissue surfaces, tissue boundaries and image texture of ordinary anatomical or pathological structures of the subject. The processor may be configured to determine the position of the ultrasonic transducer with respect to the landmarks.

In some examples, the method may include using the determined position to provide, to an operator of the apparatus, navigational guidance for movement of the imaging probe.

According to some implementations, in a non-transitory computer readable medium having software stored thereon, the software includes instructions for ultrasonography, the instructions causing an apparatus to (i) collect image data of an environment in which an ultrasonography apparatus is to be operated, the ultrasonography apparatus including one or more ultrasonic transducers, one or more inertial sensors, one or more optical sensors and a processor communicatively coupled with the one or more ultrasonic transducers, the one or more inertial sensors and the one or more optical sensors, the ultrasonography apparatus being configured to perform noninvasive medical ultrasonography; and (ii) estimate, with the processor, a spatial position of the apparatus using a combination of signals received from the one or more ultrasonic transducers, the one or more inertial sensors and the one or more optical sensors.

### BRIEF DESCRIPTION OF THE DRAWINGS

Details of one or more implementations of the subject matter described in this specification are set forth in this disclosure and the accompanying drawings. Other features, aspects, and advantages will become apparent from a review of the disclosure. Note that the relative dimensions of the drawings and other diagrams of this disclosure may not be drawn to scale. The sizes, thicknesses, arrangements, materials, etc., shown and described in this disclosure are made only by way of example and should not be construed as limiting. Like reference numbers and designations in the various drawings indicate like elements.

FIG. 1 illustrates a hand-held ultrasonic imaging probe, according to an implementation.

FIG. 2 illustrates an example of an environment in which the hand-held ultrasonic imaging probe may be operated according to an implementation.

FIG. 3 illustrates an example of a method for estimating a position of an ultrasonography apparatus, according to an implementation.

FIG. 4 illustrates an example of a method for calibrating an inertial sensor of a ultrasonic imaging probe, according to another implementation.

FIG. 5 illustrates an example of a data flow diagram according to an implementation.

FIG. 6 illustrates an example of an environment in which the hand-held ultrasonic imaging probe may be operated according to another implementation.

### DETAILED DESCRIPTION

Details of one or more implementations of the subject matter described in this specification are set forth in this disclosure, which includes the description and claims in this document, and the accompanying drawings. Other features, aspects and advantages will become apparent from a review of the disclosure. Note that the relative dimensions of the drawings and other diagrams of this disclosure may not be drawn to scale. The sizes, thicknesses, arrangements, materials, etc., shown and described in this disclosure are made only by way of example and should not be construed as limiting.

The present inventors have developed techniques for improving the portability, operability and functionality of ultrasonic scanners such that they may be used in a greater diversity of physical settings and by a user (care provider) who is not necessarily a specialized ultrasound technician (sonographer). For example, in a related provisional patent application entitled “AUTO-CONFIGURATION OF A DEVICE FOR ULTRASOUND IMAGING”, U.S. Provisional Patent Application No. 62/153,978, filed on Apr. 28, 2015, owned by the assignee of the present application, techniques are described for largely automating a process of setting up and/or optimizing settings of the ultrasonic probe. As a further example, in a related provisional patent application entitled “IN-DEVICE FUSION OF OPTICAL AND INERTIAL POSITIONAL TRACKING OF ULTRASOUND PROBES”, U.S. Provisional Patent Application No. 62/153,970, filed on Apr. 28, 2015, owned by the assignee of the present application, techniques are described that enable a hand-held ultrasonic imaging probe to determine its own spatial position using optical and inertial sensors whether or not the probe is being used in a dedicated ultrasound examination room.

The systems, methods and devices of the disclosure each have several innovative aspects, no single one of which is solely responsible for the desirable attributes disclosed herein. One innovative aspect of the subject matter described in this disclosure can be implemented in a portable ultrasonic imaging probe for medical ultrasonography. In some implementations, the portable ultrasonic imaging probe may be hand-held. In some implementations, the portable ultrasonic imaging may be included in or attached to an apparatus such as a robot, or may be or include a wearable device. For example, a sleeve, wearable by a human or robotic operator and/or by a patient or other subject of examination (hereinafter, “subject”) may contain one or more ultrasonic transducers, one or more inertial sensors, and/or one or more optical sensors.

In another example, the wearable device may contain one or more ultrasonic transducers communicatively coupled to a processor by way of a wired or wireless interface. Whether or not the wearable sleeve also includes optical sensors, the processor may also be communicatively coupled to one or more inertial sensors of the wearable device and/or one or more optical sensors disposed within an examination room where the wearable device is located. The optical sensors may be configured to capture image data of the wearable device and provide it to the processor, which can use the image data to determine a location of the wearable device. The ultrasonic transducers of the wearable device may capture ultrasound data and send it to the processor, which uses the data to generate an ultrasound volume and also determine a precise location of the wearable device relative to the subject's body.

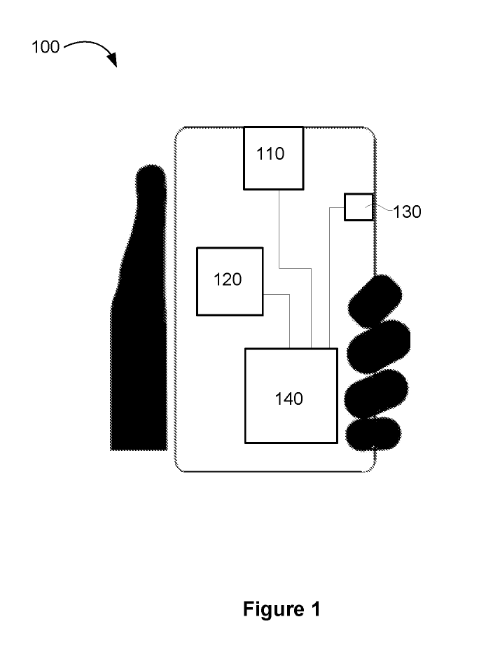

FIG. 1 illustrates a hand-held ultrasonic imaging probe, according to an implementation. The apparatus 100 includes an ultrasonic transducer 110, an inertial sensor 120, an optical sensor 130 and a processor 140 communicatively coupled with the ultrasonic transducer 110, the inertial sensor 120, and the optical sensor 130. The processor 140 may be configured to calibrate the inertial sensor 120 using outputs from the optical sensor 130. For example, the processor 140 may be configured to correct for accumulated drift errors of the inertial sensor 120. In some implementations, the hand-held ultrasonic imaging probe may be configured to make a real-time determination of its spatial position with respect to an arbitrary coordinate system using a combination of optical and inertial sensors. As used herein, and in the claims, the terms “spatial position” and “position” refers to a spatial location (e.g., in terms of X, Y and Z coordinate location) in combination with an angular orientation (e.g. roll, pitch and yaw angle) and may be referred to as a 6 degree of freedom (6-DoF) spatial position. As used herein, and in the claims, the term “optical sensor” refers to a device configured to optically detect visible, infrared and or ultraviolet light and/or images thereof, and includes any kind of camera or photodetector.

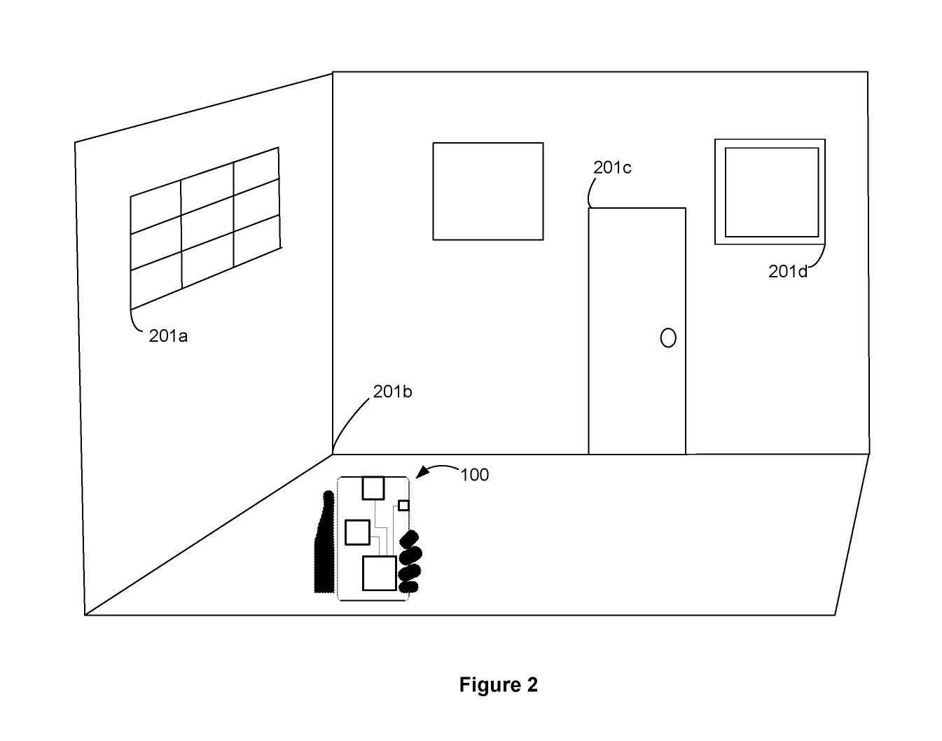

FIG. 2 illustrates an example of an environment in which the hand-held ultrasonic imaging probe may be operated according to an implementation. Where the apparatus 100 includes the processor 140 communicatively coupled with one or more optical sensors 130, the processor 140 may be configured to collect image data of an environment (for example, an examining room) in which a subject is to be examined using the apparatus. The processor 140 may be configured to process the acquired environmental image data so as to select a plurality of fixed “landmarks” in the vicinity of the probe. These landmarks may include visually well-defined points, edges or corners of surfaces, fixtures, and/or objects of an ordinary room in which an operator wishes to perform an ultrasonic exam such as corners 201a, 201b, 201c and 201d. The processor may be configured to calculate, in real time, the probe's X, Y and Z location as well as the probe's pitch, yaw, and roll orientation with respect to these landmarks. Moreover, the processor may be configured to calculate, in real time, location of the subject or an anatomical feature of the subject.

As indicated above, the processor 140 may also be communicatively coupled with at least one inertial sensor 120. The inertial sensor 120 may be configured to measure translational and rotational motion of the apparatus 100. The inertial sensor 120 may be configured as or include an accelerometer, a gyroscope, a MEMS inertial sensor, etc. Using visual inertial odometry (VIO) techniques, such as those which have been developed in the field of robotics, the processor may be configured to estimate, in real-time, the probe's spatial position notwithstanding that some or all of the landmarks 201 may be, from time to time, obscured from view of the optical sensors, and notwithstanding normal inertial sensor drift error accumulation. Alternatively, or in addition, simultaneous localization and mapping (SLAM) techniques and image registration techniques may be used. As a result, the combination of optical sensor data and inertial sensor data will enable a reasonably accurate estimation of the probe's spatial position. Thus, the estimation of the probe's position may be based on a combination of data from the inertial sensors and the optical sensors. Alternatively, or in addition, the estimation may be based on a prior position fix determined via optical sensors updated with current data from the inertial sensors.

In an implementation, the processor 140 may be configured to receive data inputs from the inertial sensor 120 and the optical sensor 130 and/or the ultrasonic transducer, and to use the received data inputs to determine the spatial position of the apparatus 100. For example, the processor may be configured to estimate a 6-DoF spatial position of the apparatus using a combination of outputs from two or more of the ultrasonic transducer 110, the inertial sensor 120 and the optical sensor 130. Moreover, the processor may be configured to correct drift error accumulation of the inertial sensor 120 using the combination of outputs. The processor 140 may be further configured to process ultrasound image data from the ultrasonic transducer 110, using the determined spatial position of the apparatus 100. For example, a series of sequential 2-D image frames (obtained for example at a rate of 30 frames per second or higher) may be collated to form a 3-D image, after appropriate adjustment of each 2-D image in view of the respective spatial position of the apparatus 100 at the time of obtaining each respective 2-D image.

In an implementation, the processor may be configured to process image data acquired by one or both of the optical sensor and the ultrasonic transducer so as to select a plurality of landmarks. As indicated above, in some implementations, the landmarks may include points, edges or corners of ordinary surfaces, fixtures, and/or objects of a room in which in which the apparatus is to be used to examine a subject. In addition, or alternatively, the landmarks may include one or more anatomical features of the subject, the anatomical features including one or more of tissue surfaces, tissue boundaries or image texture of ordinary anatomical or pathological structures of the subject.

In an implementation, the apparatus may also include one or more optical sensors that are directed towards the subject. Signals from the optical sensors may better allow the apparatus to track its position relative to the subject's body.

In another implementation, the apparatus may include one or more optical sensors directed towards the environment the apparatus is located in, and one or more optical sensors directed towards the subject. This may better allow the apparatus to determine a position of the apparatus relative to the environment and also determine the position of the apparatus to the body. As a result, even if the subject moves, the ultrasound volume generation may be substantially unimpaired because the apparatus is aware of its location with respect to the environment as well as with respect to the subject. Otherwise, if the subject moved and the apparatus only had its position relative to the environment, then the apparatus might inadvertently add ultrasound data to an incorrect ultrasound volume.

As a result, outputs of an ultrasonic scan performed by the probe may be processed, in light of the determined spatial position of the probe, to determine the relative position, in three-dimensional space, of each of a sequence of 2-D images.

In an implementation, the processor 140 may be configured to use the determined spatial position to provide, to an operator of the apparatus, navigational guidance for movement of the hand-held ultrasonic imaging probe.

Knowledge of the relative position of each 2-D image with respect to an arbitrary reference frame may enable one or more of the following applications, for example: (i) the creation of more accurate three dimensional ultrasound volumes from two-dimensional ultrasound images; (ii) the overlaying of each image onto an optical or alternative image of the subject, with accurate anatomical registration of internal structures; (iii) the combination of multiple two-dimensional images into another two-dimensional image with better quality and larger anatomical coverage, and (iv) the provision to the ultrasound operator of navigational guidance for probe movement.

Integration of the processor, the optical sensor, and the inertial sensor component as part of the hand-held ultrasonic imaging probe, enables a positional tracking function for the probe that is cost efficient and compact. The proposed techniques do not require external equipment such as magnetic trackers nor special room preparation as needed by tracking systems that rely on depth images or external vision sensors. Neither do the techniques require the application of cumbersome or conspicuous visual markers on the probe and/or the subject.

In contrast to the present disclosure, known optical-only systems demand that a large number—often hundreds—of visually conspicuous features (such as points, corners, colored patches, markers) are visible in the environment and that such features can be reliably matched between subsequent frames. Inertial sensors, on the other hand, are operable in the absence of any external visual reference, but they quickly lose absolute accuracy as the tracked device moves.

In accordance with the present disclosure, inertial sensors provide good relative positional accuracy over short periods of time during which landmarks may be obscured from the field of view of the optical sensors. This knowledge is used to accurately estimate, substantially continuously, the spatial position of the camera during an ultrasound scan. As a result, a need for a large number of specially configured conspicuous visual features in the environment of the ultrasound scan can be eliminated. Consequently, the ultrasonic imaging probe may be used to obtain real time 3-D images even in environments that have not been equipped for ultrasound imaging. For example, the present disclosure contemplates the ultrasonic imaging probe may be used in an ordinary room in which a subject may be examined such as a doctor's office, emergency room, or in a subject's home.

The application of integrated optical and inertial positional tracking is particularly apt for establishing the spatial position and orientation of ultrasound probes, because in such applications there is a reasonable expectation that the probe will be held in a particular manner by the operator, so that the optical sensors can be strategically placed on the device to ensure maximum visibility of the external environment.

The presently disclosed techniques bring many benefits to the medical diagnosis and to the user experience of the ultrasound operator and subject. In some implementations, for example, the techniques enable production of accurate three-dimensional models of a subject's anatomy and pathological structures without the use of external devices and room preparation. As a result, field application of ultrasonic imaging, outside a clinical setting may be enabled. Such 3-D models may be used in real time for a more accurate subject diagnosis or assessment, and also may be stored for future comparison against new two or three dimensional data.

As a further example, in some implementations obtained ultrasound images may be overlaid against an optical image of the subject with the appropriate anatomical alignment. Such overlay may be displayed on a separate screen or transmitted wirelessly or otherwise to a headmounted display (HMD) which would overlay the ultrasound image against a live image of the subject. In an implementation, a position of the HMD relative to the probe may be obtained and images displayed by the HMD may be adjusted based on the HMD's position relative to the probe's position. For example, the HMD may include optical and/or inertial sensors from which its 6-DoF spatial position may be obtained. Based on the obtained 6-DoF spatial position, images displayed by the HMD may be changed accordingly. For example, as an operator wearing the HMD moves around a subject's body, displayed images of the ultrasonic volume may be observed from multiple angles. In some implementations, the probe device may be a wearable sleeve with multiple ultrasonic transducers, optical and/or inertial sensors, communicatively coupled with the HMD, enabling an operator wearing the HMD to obtain a rich, three dimensional, view of a subject's anatomy or pathological structure. The multiple ultrasonic transducers, optical and/or inertial sensors may be calibrated to determine, for example, their proximity to one another prior to and/or during examination of the subject.

As a yet further example, in some implementations navigational guidance for moving the probe may be provided, with an objective of aiding the ultrasound operator in the task of placing the probe for optimal image acquisition. This may enable the use of ultrasound imaging by operators with less experience and training, thereby facilitating the adaption of ultrasound imaging technology.

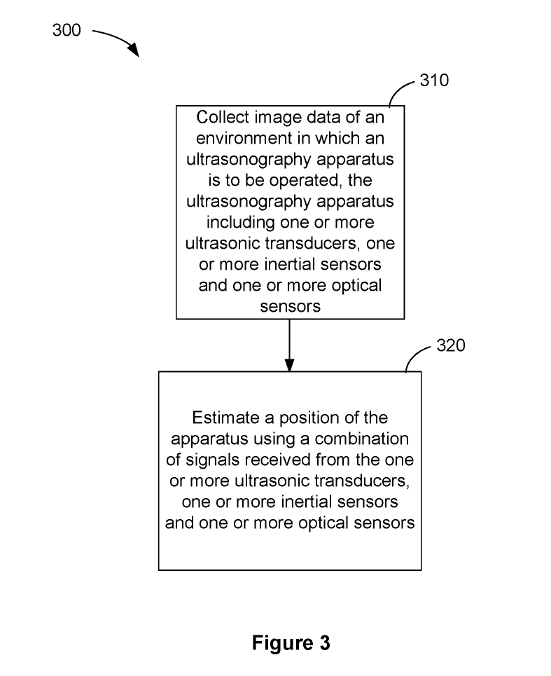

In some implementations, the integration of optical with inertial measurements may include use of an extended Kalman filter (EKF) which would optimally combine measurements from each type of sensor into an overall coherent estimation of the probes position and orientation. FIG. 3 illustrates an example of a method for estimating a position of an ultrasonography apparatus. As described hereinabove, the ultrasonography apparatus may include one or more ultrasonic transducers, one or more inertial sensors, one or more optical sensors and a processor communicatively coupled with the one or more ultrasonic transducers, the one or more inertial sensors and the one or more optical sensors. In the illustrated implementation, method 300 includes a block 310 for collecting, with the optical sensor and/or the ultrasonic transducer, image data of an environment in which the ultrasonic imaging probe is to be operated.

The method proceeds, at block 320, with estimating, using the processor, a position of the apparatus using a combination of signals received from the one or more of the ultrasonic transducers, the one or more inertial sensors and the one or more optical sensors. For example, the processor may use outputs from the optical sensor and/or the ultrasonic transducer to correct for accumulated drift errors of the inertial sensor.

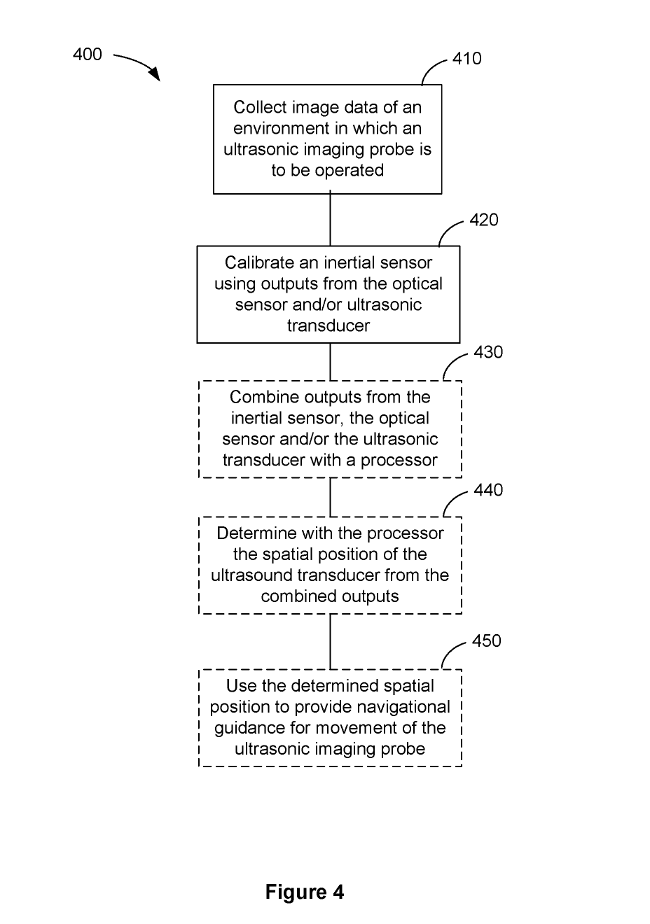

FIG. 4 illustrates an example of a method for calibrating an inertial sensor of a hand-held ultrasonic imaging probe and, according to an implementation. As described hereinabove, the imaging probe may include an ultrasonic transducer, an inertial sensor and a processor communicatively coupled with the ultrasonic transducer the inertial sensor and the optical sensor. In the illustrated implementation, method 400 includes a block 410 for collecting, with one or both of the optical sensor and the ultrasonic transducer, image data of an environment in which the ultrasonic imaging probe is to be operated.

The method proceeds, at block 420, with calibrating, using the processor, the inertial sensor, using outputs from the optical sensor and/or ultrasonic transducer. For example, the processor may use the outputs to correct for accumulated drift errors of the inertial sensor.

Optionally, in some implementations the method 400 may proceed at block 430 with combining, with the processor, outputs from the inertial sensor and from one or both of the optical sensor and the ultrasonic transducer. As a further optional step, the method 400 may proceed at block 440 with determining with the processor the spatial position of the ultrasound transducer with the combined outputs obtained at block 430. In a yet further optional step, the method 400 may proceed at block 450 with using the spatial position, determined at block 440, to provide navigational guidance for movement of the ultrasonic imaging probe. Navigational guidance may be provided to an operator using the ultrasonic imaging probe to perform noninvasive medical ultrasonography.

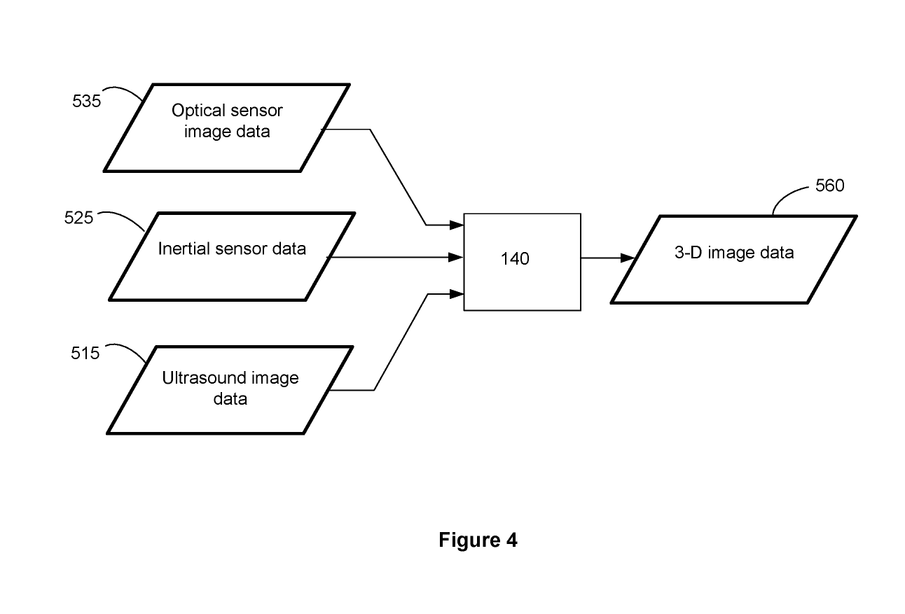

In an implementation, the processor may be configured to process ultrasound image data from the ultrasonic transducer and calibrate the estimated 6-DoF spatial position of the apparatus 100 using the processed ultrasound image data optical sensor image data. FIG. 5 illustrates an example of a data flow diagram according to an implementation. In the illustrated implementation the processor 140 processes ultrasound image data 515, inertial sensor data 525, and optical sensor image data 535. As a result, outputs from each of the ultrasonic transducer 110, the inertial sensor 120, and the optical sensor 130 may be fused so as to obtain a more accurately calibrated estimation of the spatial position of the apparatus 100.

Where the ultrasound image data 515 includes a series of 2-D image frames and the processed ultrasound image data includes a 3-D image, the processor may be configured to adjust one or more of the 2-D image frames in view of the estimated 6-DoF spatial position at the time of obtaining each respective 2-D image. For example where the estimated 6-DoF spatial position at a time corresponding to a 2-D image frame (i) is different from the estimated 6-DoF spatial position at a time corresponding to a 2-D image frame (i+1), one or both of the respective 2-D image frames may be adjusted to compensate for the difference. As a result, temporal series of 2-D images may be more accurately combined to compute 3-D image data 560.

In an implementation, the processor may be configured to make a determination whether or not an obtained 2-D image frame relates to a first volume under examination or a different volume. For example, where an operator interrupts and then resumes use of the apparatus (e.g., by lifting if up from a first location and then setting it down at a second location), the operator may or may not intend that the first location and the second location be substantially identical. Upon resumption of use of the apparatus, the processor may be configured to determine, with regard to a newly received 2-D image frame, whether data from the 2-D image frame should be merged with previously received image frame data (because the first location and the second location are substantially identical) or not merged (because the first location and the second location are not substantially identical). For example, the processor may be configured to determine a difference between two or more 2-D image frames and compare the difference to a threshold to determine if the images relate to approximately the same location. As a further example, the processor may be configured to compare the 6-DoF spatial position, as well as operator settings of the ultrasound probe (e.g., frequency and gain, image depth and signal processing filter parameters) associated with the two or more 2-D image frames to determine if they should be associated with the same volume.

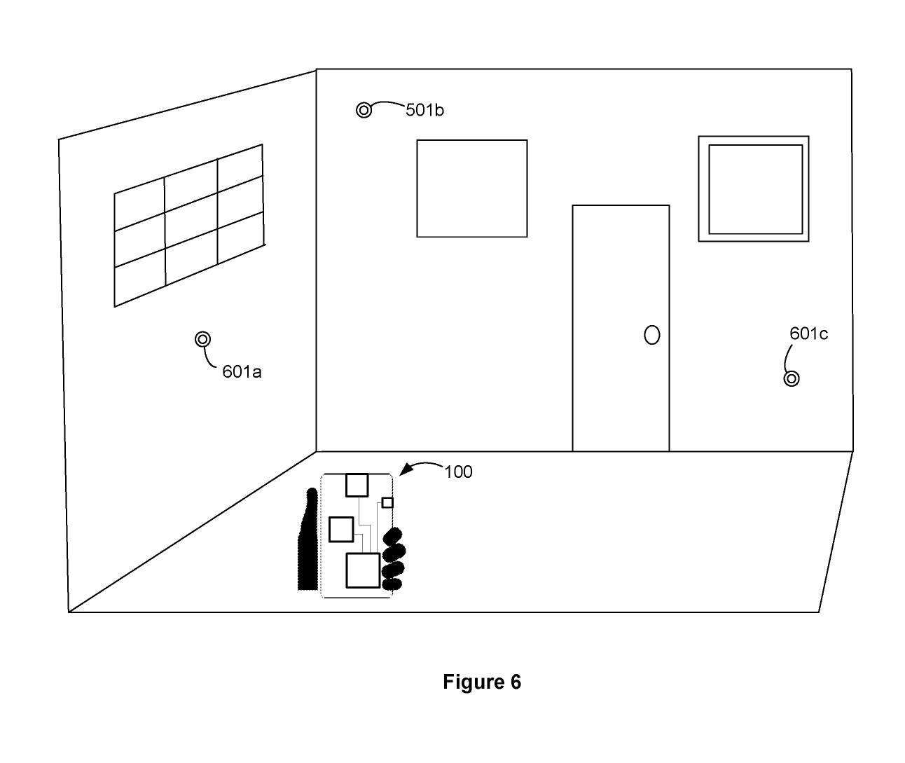

FIG. 6 illustrates an example of an environment in which the hand-held ultrasonic imaging probe may be operated according to another implementation. Where the apparatus 100 includes the processor 140 communicatively coupled with one or more optical sensors 130, the processor 140 may be configured to collect image data of an environment (for example, an examining room) in which a subject is to be examined using the apparatus. In the illustrated implementation, the examining room includes a plurality of optical emitters 501 configured for optical wireless communication (OWC). The optical sensors 130 may be optically coupled so as to receive signals from the emitters 601, which may be configured as part of an indoor positioning system (IPS). In an implementation, the optical emitters are configured for visible light communication (VLC). In other implementations, the optical emitters may be configured for communication in the infrared and/or ultraviolet light wavelengths. The IPS may enable the processor to calculate, in real time, the probe's X, Y and Z location as well as the probe's pitch, yaw, and roll orientation with respect to the optical emitters 601. Moreover, the processor may be configured to calculate, in real time, location of the subject or an anatomical feature of the subject, with or without use of an inertial sensor.

As indicated above, the processor 140 may also be communicatively coupled with at least one inertial sensor 120. The inertial sensor 120 may be configured to measure translational and rotational motion of the apparatus 100. Using VIO techniques, the processor may be configured to estimate, in real-time, the probe's spatial position notwithstanding that some or all of the optical emitters 601 may be obscured from view of the optical sensors, and notwithstanding normal inertial sensor drift error accumulation.

Thus, a smart device for ultrasound imaging has been disclosed that is configured as an ultrasonic imaging probe that includes an inertial sensor and an optical sensor where the processor is configured to calibrate the inertial sensor using outputs from the optical sensor. It will be appreciated that a number of alternative configurations and fabrication techniques may be contemplated.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor or any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by or to control the operation of data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that can be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, as a person having ordinary skill in the art will readily appreciate, the terms “upper” and “lower”, “top” and bottom”, “front” and “back”, and “over”, “on”, “under” and “underlying” are sometimes used for ease of describing the figures and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. An apparatus for ultrasonography, the apparatus comprising:
one or more ultrasonic transducers;
one or more inertial sensors;
one or more optical sensors; and
a processor communicatively coupled with the one or more ultrasonic transducers, the one or more inertial sensors and the one or more optical sensors; wherein the processor is capable of:
estimating a position of the apparatus based on a combination of signals received from the one or more ultrasonic transducers, the one or more inertial sensors and the one or more optical sensors.

2. The apparatus of claim 1, wherein the estimating the position of the apparatus comprises:
processing ultrasound image data from the one or more ultrasonic transducers; and
determining the position based on the processed ultrasound image data.

3. The apparatus of claim 2, wherein:
the ultrasound image data includes a series of 2-D image frames and the processed ultrasound image data includes a 3-D image, and
the processor is configured to adjust at least one of the 2-D image frames in view of the determined position at a time of obtaining the at least one of the 2-D images.

4. The apparatus of claim 2, wherein:
the ultrasound image data includes a series of 2-D image frames and the processed ultrasound image data includes a 3-D image of a first volume, and
the processor is configured to determine, with regard to at least one of the 2-D image frames, whether the at least one of the 2-D image frames relates to the first volume or to a different volume.

5. The apparatus of claim 1, wherein the optical sensor is optically coupled with one or more optical wireless communication (OWC) emitters of an indoor positioning system.

6. The apparatus of claim 1, wherein the processor is configured to correct drift error accumulation of the inertial sensors using the combination of signals.

7. The apparatus of claim 1, wherein the processor is configured to process image data acquired by one or both of the optical sensors and the ultrasonic transducers so as to select a plurality of landmarks.

8. The apparatus of claim 7, wherein the landmarks include one or both of:
one or more points, edges or corners of ordinary surfaces, fixtures or objects of a room in which the apparatus is to be used to examine a subject; and
one or more anatomical features of the subject, the anatomical features being selected from the group consisting of tissue surfaces, tissue boundaries and image texture of ordinary anatomical or pathological structures of the subject.

9. The apparatus of claim 8, wherein the processor is configured to calculate the position of the apparatus with respect to the landmarks.

10. The apparatus of claim 9, wherein the processor is configured to calculate a location of the subject or an anatomical feature of the subject.

11. The apparatus of claim 1, wherein the processor is configured to fuse the combination of signals using one or more of visual inertial odometry (VIO) techniques, simultaneous localization and mapping (SLAM) techniques, image registration techniques, or any combination thereof.

12. The apparatus of claim 11, wherein the processor is configured to:
process ultrasound image data from the ultrasonic transducer; and
make a determination of the position of the apparatus from the processed ultrasound image data.

13. The apparatus of claim 12, wherein the processor is configured to use the determination to provide, to an operator of the apparatus, one or more of: navigational guidance for movement of the imaging probe, notifications based on the determination, identification of anatomical features, identification of pathological structures or any combination thereof.

14. A method for ultrasonography, the method comprising:
collecting image data of an environment in which an ultrasonography apparatus is to be operated, the ultrasonography apparatus including one or more ultrasonic transducers, one or more inertial sensors, one or more optical sensors and a processor communicatively coupled with the one or more ultrasonic transducers, the one or more inertial sensors and the one or more optical sensors, the ultrasonography apparatus being configured to perform noninvasive medical ultrasonography; and
estimating, with the processor, a position of the apparatus using a combination of signals received from the one or more ultrasonic transducers, the one or more inertial sensors and the one or more optical sensors.

15. The method of claim 14, further comprising:
fusing, with the processor, the combination of signals using one or more of visual inertial odometry (VIO) techniques, simultaneous localization and mapping (SLAM) techniques, image registration techniques, or any combination thereof.

16. The method of claim 14, wherein:
the image data includes outputs from one or both of the optical sensors and the ultrasonic transducers;
the processor is configured to process the image data so as to select a plurality of landmarks, the landmarks including one or both of:
one or more points, edges or corners of ordinary surfaces, fixtures or objects of a room in which the apparatus is to be used to examine a subject; and
one or more anatomical features of the subject, the anatomical features being selected from the group consisting of tissue surfaces, tissue boundaries and image texture of ordinary anatomical or pathological structures of the subject; and
the processor is configured to determine the position of the ultrasonic transducer with respect to the landmarks.

17. The method of claim 14, further comprising using the determined position to provide, to an operator of the apparatus, navigational guidance for movement of the imaging probe.

18. A non-transitory computer readable medium having software stored thereon, the software including instructions for ultrasonography, the instructions causing an apparatus to:
collect image data of an environment in which an ultrasonography apparatus is to be operated, the ultrasonography apparatus including one or more ultrasonic transducers, one or more inertial sensors, one or more optical sensors and a processor communicatively coupled with the one or more ultrasonic transducers, the one or more inertial sensors and the one or more optical sensors, the ultrasonography apparatus being configured to perform noninvasive medical ultrasonography; and
estimate, with the processor, a spatial position of the apparatus using a combination of signals received from the one or more ultrasonic transducers, the one or more inertial sensors and the one or more optical sensors.

19. The computer readable medium of claim 18, wherein the processor is configured to correct drift error accumulation of the inertial sensors using the combination of signals.

20. The computer readable medium of claim 18, wherein:
the image data includes outputs from one or both of the optical sensors and the ultrasonic transducers;
the processor is configured to process the image data so as to select a plurality of landmarks, the landmarks including one or both of:
one or more points, edges or corners of ordinary surfaces, fixtures or objects of a room in which the apparatus is to be used to examine a subject; and
one or more anatomical features of the subject, the anatomical features being selected from the group consisting of tissue surfaces, tissue boundaries and image texture of ordinary anatomical or pathological structures of the subject; and
the processor is configured to determine the position of the apparatus with respect to the landmarks.
